SEQUENCE LISTING (2 sequences)

SEQ ID NO: 1 - DNA, 3822 bp, coronavirus
  misc_feature (1)..(3822): coronavirus 2 isolate Wuhan-Hu-1
atgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataa

SEQ ID NO: 2 - protein, 1273 aa, coronavirus
  MISC_FEATURE (1)..(1273): coronavirus 2 isolate Wuhan-Hu-1
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

(19) United States
(12) Patent Application Publication
(10) Pub. No.: US 2023/0398206 A1
(43) Pub. Date: Dec. 14, 2023

(54) METHOD AND KIT FOR DETECTION OF CELL MEDIATED IMMUNE RESPONSE

(71) Applicant: BIOVAXYS INC., New York, NY (US)

(72) Inventors: David Berd, Jenkintown, PA (US); James Passin, New York, NY (US)

(21) Appl. No.: 18/250,694

(22) PCT Filed: Oct. 27, 2021
(86) PCT No.: PCT/US2021/056759

§ 371 Date: Apr. 26, 2023

Related U.S. Application Data
(60) Provisional application No. 63/106,480, filed on Oct. 28, 2020.
(60) Provisional application No. 63/106,482, filed on Oct. 28, 2020.

(51) Int. Cl. A61K 39/215; C07K 14/165

(52) U.S. Cl. CPC A61K 39/215; C07K 14/165; A61K 2039/54

(57) ABSTRACT

The invention relates to a method of eliciting an immune response in a human subject showing no signs or symptoms of an active SARS-CoV-2 infection comprising administering intra-dermally to the human subject an immunogenic composition comprising the Spike protein (S protein) of SARS-CoV-2 or a fragment thereof.

### SEQUENCE LISTING SUBMISSION VIA EFS-WEB

A computer readable text file, entitled “SequenceListing.txt,” created on or about Jan. 6, 2020 with a file size of about 4 kb contains the sequence listing for this application and is hereby incorporated by reference in its entirety.

### FIELD OF THE INVENTION

This invention relates to an in vivo diagnostic method of detecting a cell mediated immune response to one or more specific SARS-CoV-2 antigens in a human or animal.

### BACKGROUND OF THE INVENTION

Delayed type hypersensitivity (DTH) is the oldest test of human T lymphocyte function. It was first demonstrated by Robert Koch, the discoverer of the bacterium that causes tuberculosis (Black (1999) Dermatology Online Journal, 5: 7-25). He serendipitously found that the intradermal injection of killed M. tuberculosis induced a cutaneous inflammatory response in patients who had previously been exposed to the organism. It soon became apparent that what was known as “cutaneous sensitivity” comprised several varieties of reactions. Immediate reactions, which developed within minutes or several hours of skin injection, were mediated by antibody, while delayed reactions, which required 24-72 hours to develop, were mediated by cells (Janeway (2001) Immunobiology, pp. 471-500, Garland Publishing).

The importance of immunocompetent cells to delayed reactions was first demonstrated in a landmark paper by Landsteiner and Chase. They sensitized guinea pigs to picryl chloride (trinitrochlorobenzene) and then harvested their peritoneal exudate cells. Transfer of these cells, but not the supernatant obtained after centrifugation, to naïve guinea pigs rendered the recipients sensitive to picryl chloride. Moreover, this response required living cells, since it was abrogated if the cells were killed by heating prior to the transfer. Understanding of this phenomenon was further refined by Scovern and Kantor, who showed that the cells capable of transferring DTH were T lymphocytes. Moreover, experiments testing serial dilutions of immune cells have shown that transfer of a single antigen-specific T cell is sufficient to elicit a DTH reaction in naïve mice (Marchal (1982) J. Immunol. 129: 954-958). Conversely, the overwhelming majority of T cells isolated from a DTH reaction are not specifically sensitized, so it is apparent that nonspecific inflammation augments the initial specific reaction (Najarian (1963) J. Exp. Med. 118: 341-352).

These observations subsequently were translated to a human system: Injection of human T-lymphocyte enriched peripheral blood lymphocytes into a mouse foot pad induced antigen-specific DTH reactions to Candida organisms or to trinitrophenyl (TNP)-modified autologous lymphocytes (Trial (1989) Reg. Immunol. 2: 14-21). The hallmark of a DTH reaction is the development of induration and erythema 24-72 hours after intradermal injection of antigen. Histologic studies have been conducted in humans known to exhibit DTH to purified protein derivative (PPD) (Poulter (1982) Cell. Immunol. 74: 358-369). Intradermal injection of PPD induces perivascular accumulation of T lymphocytes by 12 hours, which increases by 24 hours and peak at 48 hours. Some of the T cells diffuse throughout the dermis. CD4+ T cells are the majority with a CD4+/CD8+ ratio of about 2:1, although CD8+ T cells may constitute the majority in the response to other antigens (Puccetti (1994) Eur. J. Immunol. 24: 1446-1452). The T cells become progressively activated, as shown by detection of the IL2 receptor on 20% of them by 48 hours (Platt (1983) J. Exp. Med. 158: 1227-1242). Also prevalent in the dermis are activated macrophages and dendritic cells; B lymphocytes are absent. Control subjects, i.e., PPD (−) individuals, do not develop clinically apparent reactions; however, biopsy of their injection sites shows limited perivascular accumulation of mononuclear cells without any evidence of T cell activation.

Functional analyses of T cells comprising DTH reactions to PPD or Candida antigen were performed using the “skin window” technique (MacPhee (1993) Cell. Immunol. 151: 80-96). Cell-rich exudates were withdrawn, and antigen-reactive cells were cloned at limiting dilution. Both CD4+ and CD8+ T cell clones were obtained, and the cytokine production pattern (TNF, IL2, gamma interferon) was characteristic of type I T cells. Skin chamber fluids contained high concentrations of gamma interferon and TNF. The appropriate controls were negative: these cytokines were not detectable in the fluids from skin chambers placed over sites injected with an antigen that did not elicit DTH.

The inflammatory responses described above cause swelling of endothelial cells lining dermal venules, which become leaky to macromolecules. Fibrinogen that leaks into the dermis is cleaved to fibrin which in turn traps proteins and forms a dermal gel (Ahmed (1983) Arch. Dermatol. 119: 934-945). This is the cause of the dermal edema seen microscopically and the induration felt by the examiner, which characterize the classical DTH reaction.

Medical Uses of DTH Testing are known. The elicitation of DTH by intradermal injection of “tuberculin” has been used as a test for exposure for tuberculosis since Koch's original observation over a hundred years ago. Tuberculin, a concentrated filtration of culture supernatants of M. tuberculosis, has been long since replaced by PPD, an ammonium sulfate precipitation of the supernatant. At least 95% of patients exposed to tuberculosis have a positive DTH response to PPD (Ahmed (1983) Arch. Dermatol. 119: 934-945). False positives occur mainly in patients infected with other mycobacteria. False negatives are seen in immunosuppressed subjects, e.g., those infected with HIV or taking corticosteroids. The reliability of the PPD test, however, is demonstrated by the fact that the decision to treat a patient for tuberculosis is made on the basis of the results (Jasmer (2002) N. Engl. J. Med. 347: 1860-1866).

The PPD is a semi-quantitative test, but there is a universally accepted cutoff for a positive: ≥5 mm diameter of induration. Positive reactions are usually characterized by erythema as well, but only the diameter of induration is counted. Patients who have active tuberculosis usually have larger reactions—≥15 mm. DTH reactions between 5 mm and 10 mm may indicate active infection in immunosuppressed patients or infections that occurred in the distant past.

Fungal Diseases—DTH testing is used to assist in the diagnosis of histoplasmosis and coccidioidomycosis. A positive DTH reaction to histoplasmin indicates that a patient has been exposed to histoplasmosis, and a negative test strongly suggests the contrary (Buechner et al). The same is true for coccidioidomycosis. Moreover, the ability to generate a positive DTH reaction to coccidioidin is strongly correlated with prognosis; infected patients with negative tests have markedly lower survival rates (Ahmed (1983) Arch. Dermatol. 119: 934-945; Drutz (1978) Amer. Rev. Resp. Dis. 117: 559-585).

Viral Infections—Mumps skin test antigen is a suspension of killed virus. About 75% of subjects who subsequently developed mumps infection had negative DTH tests. Moreover, there was a strong inverse correlation between the intensity of the DTH reaction and the susceptibility to infection (Enders et al). Because most adults have had either clinically evident or subclinical infection with mumps virus, the DTH test is used today mainly as a measure of the integrity of the cell-mediated immune system.

### SUMMARY OF THE INVENTION

The invention encompasses a method of eliciting an immune response in a human subject showing no signs or symptoms of an active SARS-CoV-2 infection comprising administering intra-dermally to the human subject an immunogenic composition comprising the Spike protein (S protein) of SARS-CoV-2 or a fragment thereof. In some embodiments, the method further comprises measuring the magnitude of induration and erythema in the skin at the site of the injection. In some embodiments, the magnitude of induration is measured between twenty-four to seventy-two hours after administration of the immunogenic composition.

In some embodiments, the mangnitude of induration in the skin is measured by cutaneous ultrasound. In some embodiments, infra-red or thermal images are used to measure the magnitude of induration in the skin using a Therma CAM SC 500 thermovision camera under standard conditions for diagnostic thermal imaging as set forth in Mikulska (2005) Thermology International 15: 134-139. In some embodiments, the magnitude of the induration in the skin is measured by using a software tool executed by a hardware processor of a computer device, or a digital or analog optical image processor wherein the optical or computer device is configured to acquire at least one image of the skin at the site of the injection. In some embodiments, the computer device is a mobile device. In some embodiments, at least one image is analyzed at least in part by the hardware processor to automatically generate at least one indicator of a plurality of indicators each representing a respective probability of the human subject having SARS-CoV-2 infection, having had exposure to SARS-CoV-2, or having been vaccinated against SARS-CoV-2. In some embodiments, the plurality of indicators comprise a plurality of scores.

In some embodiments, the fragment thereof is the 51 subunit of the S protein. In some embodiments, the fragment thereof is the S2 subunit of the S protein. In some embodiments, the S protein comprises SEQ ID NO: 2. In some embodiments, the 51 subunit comprises amino acids 17-680 of SEQ ID NO: 2. In some embodiments, the S2 subunit comprises amino acids 727-1195 of SEQ ID NO: 2. In some embodiments, the SARS-CoV-2 S protein immunogen is a fragment comprising amino acids at positions 417-560 of SEQ ID NO:2 corresponding to the receptor binding domain (RBD), or a portion thereof, may be immunogenic and an immune response specific for one or more epitopes. In some embodiments, the S protein or fragment thereof is administered as a fusion protein. In some embodiments, the S protein or fragment thereof is conjugated to a hapten.

In some embodiments, the immunogenic composition comprises at least one excipient. In some embodiments, the at least one excipient comprises phosphate buffered saline (PBS) with 0.01% Polysorbate-20 and 0.5% phenol. In some embodiments, the S protein or fragment thereof is present in an amount of 5-50 μg/mL. In some embodiments, the immunogenic composition comprises one or more additional SARS-CoV-2 proteins selected from the group consisting of M protein, N protein and E protein. In some embodiments, the human subject has been administered a vaccine to prevent SARS-Cov-2 infection. In some embodiments, the human subject has previously been infected with SARS-CoV-2. In some embodiments, the human subject previously suffered from symptoms of Covid-19. In some embodiments, the human subject has not previously suffered from any symptoms of Covid-19.

The invention also encompasses a kit comprising an immunogenic composition comprising S protein from SARS-CoV-2 and means for intradermal injection of the immunogenic composition.

### DETAILED DESCRIPTION OF THE INVENTION

The present invention provides a method for specific diagnosis of previous or ongoing coronavirus infection, in a human subject, the method comprising intradermally injecting, in the human subject, a composition containing a polypeptide which comprises a Spike protein (S protein) from SARS-CoV-2 with an amino acid sequence which has a degree of sequence identity of at least 95% with SEQ ID NO: 2, or a subunit or fragment thereof which comprises at least one T-cell epitope, with which lymphoid T-cells previously primed with S protein SARS-CoV-2 are capable of reacting in vivo, e.g. manifested as a positive Delayed-Type Hypersensitivity (DTH) reaction, and with which lymphoid T-cells previously primed with an S proteins from other coronaviruses are incapable of reacting, a subsequent positive skin response at the location of injection being indicative of the human subject having and/or having had a SARS-CoV-2 infection or immunization with a SARS-Co-2 vaccine, and a negative skin response at the location of injection being indicative of the human subject not having and/or having had a SARS-CoV-2 infection. The immunogenic composition is not limited to the S protein of SARS-CoV-2. As such, variants of the SARS-CoV-2 are encompassed in the invention, including the alpha, beta and delta variants of SARS-CoV-2. In addition, any of the other proteins of SARS-CoV-2 may be utilized, including the Membrane (M) protein, Envelope (E) protein or Nucleocapsid (N) protein may be utilized, including combinations thereof. Also, the immunogenic composition could include inactivated or attenuated whole virus.

The present invention also provides a kit when used for diagnosing a SARS-CoV-2 infection comprising as one part of the kit an immunogenic agent for intradermal injection comprising S protein from SARS-CoV-2 with an amino acid sequence which has a degree of sequence identity of at least 95% with SEQ ID NO: 2, a subunit or fragment thereof which comprises at least one T-cell epitope, digital means for detecting a positive skin response at the location of injection being indicative of a human subject having or having had a coronavirus infection, or being vaccinated with a SARS-CoV-2 S protein immunogenic composition, and a negative skin response at the location of injection being indicative of the human subject not having or not having had a SARS-CoV-2 infection.

The present invention also provides a method for vaccinating a human subject against SARS-CoV-2 and to monitor immune status of the human subject with respect to this coronavirus, the method comprising (a) vaccinating the human subject against coronavirus with an immunogenic agent, and (b) prior to vaccination or periodically after vaccination assessing the human subject's immune status with respect to coronavirus by means of the method comprising intradermally injecting, in the human subject, a composition containing a polypeptide which comprises an S protein from SARS-CoV-2 with an amino acid sequence which has a degree of sequence identity of at least 95% with SEQ ID NO: 2, or a subunit or fragment thereof which comprises at least one T-cell epitope, with which lymphoid T-cells previously primed with S protein SARS-CoV-2 are capable of reacting in vivo, e.g. manifested as a positive Delayed-Type Hypersensitivity (DTH) reaction, and with which lymphoid T-cells previously primed with an S proteins from other coronaviruses are incapable of reacting, a subsequent positive skin response at the location of injection being indicative of the human subject having and/or having had a SARS-CoV-2 infection or immunization with an S protein based vaccine, and a negative skin response at the location of injection being indicative of the human subject not having and/or having had a SARS-CoV-2 infection.

As used herein, the term “immunogenic agent” encompasses any substance, composition of matter, or composition of organic material as for example a suspension of cells or cell components, the immunogenic agent being capable of conferring a substantial immune response to a coronavirus in a human subject, when administered in a suitable amount and in admixture with suitable substances.

In an alternative embodiment of the invention, T-cells primed with this immunogenic agent should not react with a polypeptide capable of reacting with T-cells primed with coronavirus other than SARS-CoV-2. The existence of an immunogenic agent comprising a SARS-CoV-2 S protein, subunit or fragment thereof has for the first time been demonstrated by intradermal injection to detect human subjects who have been vaccinated against SARS-CoV-2 and individuals who have been infected with SARS-CoV-2.

An example of an immunogenic agent with the properties described above is SEQ ID NO: 2 comprising the S protein amino acid sequence from SARS-CoV-2. Together with the S protein from SEQ ID NO: 2, other amino acid sequences for strains of SARS-CoV-2 S protein include any of those disclose in Deng (2020) Science, 8: eabb9263 and Taboada (2020) J. Virol. 94: e01056. However, it is highly likely that other SARS-CoV-2 strains will exhibit substantially the same immunological properties as SEQ ID NO: 2 and S proteins, fragments and subunits thereof from such strains for intradermal injection are also a part of the method and kit according to the invention. Other examples include the M protein, E protein or N protein from SARS-CoV-2, including combinations thereof.

As used herein, the term “polypeptide” encompasses both short peptides with a length of at least two amino acid residues and at most 10 amino acid residues, oligopeptides (11-100 amino acid residues), and longer peptides (the usual interpretation of polypeptide, i.e. more than 100 amino acid residues in length) as well as proteins (the functional entity comprising at least one peptide, oligopeptide, or polypeptide which may be chemically modified by glycosylation, or conjugated to other chemical groups). The definition of polypeptides also comprises native forms of polypeptides or proteins in SARS-CoV-2 as well as recombinant proteins or peptides in any type of expression vectors transforming any kind of host, an also chemically synthesized peptides.

Accordingly, another aspect of the invention is a method of diagnosing SARS-CoV-2 infection (active or previous infection) caused by any strain of SARS-CoV-2 in a human subject, comprising intradermally injecting, in the human subject, an immunogenic composition containing a polypeptide with which lymphoid cells previously primed with S protein from SARS-CoV-2 are capable of reacting, or a variant which is immunologically equivalent to the polypeptide, a positive skin response at the location of injection being indicative of the human subject having or having had a SARS-CoV-2 infection, and a negative skin response at the location of injection being indicative of the human subject not having or not having had a SARS-CoV-2 infection.

A further aspect of the invention is an amino acid fragment comprising a subsequence or an analog or a variant of the S protein amino acid sequence shown in SEQ ID NO: 2, the subsequence (including subunit 1 or subunit 2), an analog or variant polypeptide which is immunologically equivalent to the polypeptide in SEQ ID NO: 2.

By the terms “analog” or “variant” with regard to the polypeptide fragments of the invention is intended to indicate an amino acid sequence of a polypeptide exhibiting identical or substantially identical immunological properties to a polypeptide fragment shown in SEQ ID NO: 2. Therefore, the terms “analog” and “variant” are used in the present context to indicate a polypeptide fragment or an amino acid sequence of a similar polypeptide or amino acid sequence as the amino acid sequence constituting SARS-Co-2 S protein, allowing for minor variations which do not have an adverse effect on the ligand binding properties and/or biological function and/or immunogenicity as compared to the native S protein, or which give interesting and useful novel binding properties or biological functions and immunogenic properties of the analog. The analogous polypeptide fragment or amino acid sequence may be derived from an animal or a human or may be partially or completely of synthetic origin as described above. The analog may also be derived through the use of recombinant DNA techniques. Furthermore, the terms “analog” and “subsequence” are intended to allow for variations in the amino acid sequence such as substitution, insertion (including introns), addition, deletion and rearrangement of one or more amino acids, which variations do not have any substantial effect on the polypeptide or a subsequence thereof.

The term “substitution” is intended to mean the replacement of one or more amino acids in the full amino acid sequence with one or more different nucleotides, “addition” is understood to mean the addition of one or more amino acids at either end of the full amino acid sequence, “insertion” is intended to mean the introduction of one or more amino acids within the full amino acid sequence, “deletion” is intended to indicate that one or more amino acids have been deleted from the full amino acid sequence whether at either end of the sequence or at any suitable point within it, and “rearrangement” is intended to mean that two or more amino acids have been exchanged with each other.

In yet another aspect, the invention relates to a polypeptide having an amino acid sequence comprising a subsequence, an analog or a variant of the S protein amino acid sequence shown in SEQ ID NO: 2, the polypeptide being immunologically equivalent to the polypeptide having the amino acid sequence shown in SEQ ID NO: 2. A subsequence of the S protein is encompassed in the invention, a subsequence which encodes a T-cell epitope responsible for the elicitation of the immunological response which can be identified in a skin test. Thus, the polypeptide are embodied subsequences of the proteins of the invention, as are analogs and variants of the polypeptide subsequence. Of course, also any nucleotide sequence encoding this fragment, as well as analogs and subsequences encoding this polypeptide subsequence are embodiments of the invention.

In the present context the term “immunologically equivalent” means that the polypeptide is functionally equivalent to the polypeptide having the amino acid sequence shown in SEQ ID NO: 2 with respect to its ability of eliciting a DTH reaction to an extent of at least 45% of the DTH reaction elicited by the polypeptide under the same conditions, such as at least 65%, such as at least 85%, measured as the diameter of induration of the DTH reaction.

Furthermore, the present invention relates to a method of vaccinating one or more selected human subjects of a population against SARS-CoV-2 and subsequently subjecting the population to diagnostic tests for SARS-CoV-2, comprising vaccinating the human subjects with a vaccine, which comprises as its effective component the above-discussed S protein immunogenic agent, and subsequently subjecting the population to intradermal injection of an immunogenic composition containing a polypeptide with which lymphoid cells previously primed with S protein from the vaccine are capable of reacting in vivo, whereby a positive skin induration response at the location of injection is indicative of the human subject having either been vaccinated or previously infected with SARS-CoV-2, and a negative skin response at the location of injection is indicative of the human subject not having SARS-CoV-2, and to the use of a vaccine, which comprises as its effective component the above-discussed immunogenic agent (S protein of SARS-CoV-2).

In the method of the invention, there is no risk that the vaccination interferes with the skin testing for diagnosing previous SARS-CoV-2 infection or vaccination because with the S protein used for the vaccine and the diagnostic skin test, positive DTH reactions will only occur in human subjects previously infected with SARS-CoV-2, whereas no DTH reaction can be observed in human subjects previously vaccinated and not suffering from Covid-19 or testing negative for SARS-CoV-2.

The importance of the method of the invention is the ability of eliciting a delayed type hypersensitivity reaction in human subjects having active or previous SARS-CoV-2 infection associated with expression of the S protein from SARS-CoV-2. The DTH reaction is an inflammatory reaction occurring in the dermal environment exhibiting the cardinal features of erythema and induration due to cellular infiltration and edema. The diameter of this reaction is measured by visual inspection and also includes digital imaging of the reaction and means to assess the scope of the reaction by measuring the size of erythemia and induration. The kit according to the invention will thus be useful in assessing a human subject's immune status with respect to SARS-CoV-2 before vaccination, either to diagnose previous infection or to diagnose that the human subject has been vaccinated with a vaccine different from the vaccine defined above.

Furthermore, the kit may be useful for vaccinating individuals of a population and subsequently following their immune status with respect to coronavirus infections. The immunization caused by the vaccination may be associated with a positive response to the skin test. Vaccinated human subjects may be subjected to the skin test periodically, such as every one to three months, but other intervals may also be suitable depending on the population to be tested.

In the present context the wording “immune status with respect to SARS-CoV-2” means whether the human subject in question has a positive or negative immune response, when measured with the skin test of the present invention, which skin test is specific for SARS-CoV-2 infection and therefore gives a specific picture of their immune status, i.e. whether they have coronavirus or not.

The kit may comprise several skin tests, such as three, four or five skin tests, whereby the kit may be used for several years after the vaccination. Furthermore, by using the above defined method of diagnosing SARS-CoV-2 infection, it is thus possible to follow disease transmission rate by skin testing surveys in populations by subjecting the human subjects of the population to a diagnostic skin test as defined above or as a diagnostic tool in individual cases, and thereby diagnose the human subject(s) suffering from active Covid-19 without having positive results from human subjects previously vaccinated and not having active Covid-19. Skin tests in human subjects suffering from Covid-19 may be negative due to the absence of an immune response in critically ill subjects. DTH is frequently tested clinically with the use of skin tests to recall antigens such as purified protein derivative (PPD), tetanus toxoid, candida and other antigens. One test device, the Merieux Multitest CMI device, is used for assessment of delayed cutaneous hypersensitivity using seven standardized antigens. In some embodiments, the kit includes a similar device were DTH can be tested clinically with the use of skin tests to simultaneously recall multiple antigens related to SARS-CoV-2 in distinct areas of administration. In some embodiments, different SARS-CoV-2 proteins or fragments thereof could be administered to the skin simultaneously in different areas using a multi-prong device like the Merieux Multitest CMI device.

Thus, the S protein is one that is capable of reacting with lymphoid cells that previously have been primed with S-protein from SARS-CoV-2. In an alternative embodiment, the S protein is one that is not capable of reacting with lymphoid cells that previously have been primed with coronavirus from the above-discussed immunogenic agent.

A method of measuring cellular immunity against the polypeptide, i.e. measuring whether the polypeptide reacts with lymphoid cells previously primed may be carried out either in an in vitro system or an in vivo system. One in vitro system may be a lymphocyte proliferation assay. In this assay peripheral blood monocytes from human subjects vaccinated with a vaccine comprising as its effective component the above-discussed immunogenic agent and from human subjects having or had a SARS-CoV-2 infection are co-cultured for 4 to 5 days in the presence of the polypeptide as antigen. Immune lymphoid cells will proliferate in response to the antigenic stimulus and the proliferation is quantitated by the addition to the culture of ³H-thymidine which will be incorporated in the DNA during cell replication and measuring the amount of ³H-thymidine. An in vivo system may be measurement of the DTH reaction occurring about 24 to 48 hours after intracutaneous or intradermal injection of S protein antigen in a human subject or animal.

In the present context the term “immunologically equivalent variant, analog or subsequence” means a variant, analog or subsequence of the S protein, which is capable of reacting with lymphoid cells primed as described above and eliciting responses which are substantially identical to the responses elicited by the polypeptide itself.

When the kit and the method of diagnosing SARS-CoV-2 is used, the skin response should be measured a few days after the intradermal injection has been performed. The skin response mostly appears 1-4 days after the injection, such as 2-3 days. If a skin response is observed and has waned before 24 hours have passed after the injection, it is mostly due to an irrelevant reaction which is not indicative of the human subject having or had a SARS-CoV-2 infection. The skin response is measured as described above by visual inspection and by the use of a ruler. A positive skin response is mostly between 0.5 cm and 4.0 cm in diameter, more often between 1.0 cm and 3.0 cm in diameter.

In embodiments, methods in accordance with the present disclosure comprise measuring the magnitude of at least one of induration and erythema in the skin at the site of injection of an immunogenic composition. The magnitude of induration and erythema, and other changes to the skin (sometimes collectively referred to herein as a “skin response”) can be measured manually, automatically, or using a combination thereof.

In some embodiments, when the kit and the method of diagnosing SARS-CoV-2 are used, the skin response is measured a few days after the intradermal injection has been performed. The skin response mostly appears 1-4 days after the injection, such as 2-3 days. If a skin response is observed and has waned before 24 hours have passed after the injection, it is mostly due to an irrelevant reaction which is not indicative of the human subject having or had a SARS-CoV-2 infection.

In some embodiments, the skin response is measured as described above by visual inspection and by the use of a measuring instrument such as a ruler. In embodiments, a positive skin response is between about 0.5 cm and about 4.0 cm in diameter, more often between about 1.0 cm and about 3.0 cm in embodiments, a positive skin response is between about 0.5 cm in diameter. In some embodiments, a positive skin response is from about 0.5 cm to about 4.0 cm in the largest dimension of an induration of a skin's reaction to injection of an immunogenic composition, e.g., from about 0.5 cm to about 3.5 cm, or from 0.5 cm to about 3.0 cm, or from 1.0 cm to about 3.0 cm, or from about 1.5 cm to about 3.0 cm in the largest dimension of the induration. In some embodiments, the largest dimension of the induration that is between about 1.0 cm and about 3.0 is indicative of a positive skin response.

In embodiments, a positive skin response can additionally be determined based on other properties of the skin response, including but not limited to skin color (e.g., a change in the skin color), shape of the response area, how quickly the response developed, etc.

In some embodiments, the magnitude and other properties of the skin response, such as, without limitation, the magnitude of induration, are measured using a computer device that employs digital image processing. The described approach has an improved sensitivity, specificity, and accuracy of diagnosis as compared to visual analysis of images by a human.

The computer device can be a mobile device or it can be any other device(s) that can acquire digital images of the subject's skin at a location of an injection of an immunogenic composition. In some embodiments, the device is a mobile device (e.g., a smartphone, a tablet, smart glasses, smart watch, etc.) having a camera configured to acquire images. The images can be still or video images. The device comprises a hardware processor and hardware memory storing computer-executable instructions that, when executed by the processor, cause the device to acquire at least one image of the subject's skin. The device can have a computer software tool (e.g. an “app”) installed and executed thereon, that allows the device to be used in diagnosing SARS-CoV-2 in accordance with embodiments of the present disclosure. The app can communicate over a network with a server.

In embodiments, acquired digital image information is used to automatically (i.e. without human intervention) determine one or more properties of the image information to generate a prediction regarding the subject having or having had a SARS-CoV-2 infection or the subject having been vaccinated against SARS-CoV-2. The properties can be converted into indicators that allow assessing a SARS-CoV-2 status of a subject. In some embodiments, the one or more determined properties can be represented as scores, such that the computer device can provide one or more scores indicative of a detection of an exposure to SARS-CoV-2 and/or representing a severity of the current or prior infection. For example, in an embodiment, a plurality of scores can be used, such that a lowest score is indicate of a low probability of exposure to SARS-CoV-2 (e.g., smaller than about 10%, or smaller than about 5%, or smaller than about 1%), whereas the highest score is indicative of a high probability of exposure to SARS-CoV-2 (e.g., greater than about 70%, or greater than about 80%, or greater than about 90%, or greater than about 95%, or greater than about 96%, or greater than about 97%, or greater than about 98%, or greater than about 99%, or about 99%).

In some embodiments, a suitable number of scores can be used, e.g. scores from 1 to 5, or from 1 to 4, or from 1 to 3, or 1 and 2, wherein the lowest score can be representative of a low probability of exposure to SARS-CoV-2, and the highest score can be representative of a high probability of exposure to SARS-CoV-2. The one or more scores that are intermediate (between the lowest and the highest) are representative of respective degrees of severity of the exposure to SARS-CoV-2. A zero score can also be used in some cases, e.g., to represent that a prior or current exposure to SARS-CoV-2 has not been detected. In some embodiments, the scores each represent a likelihood of a prior or current exposure to SARS-CoV, determined based on image analysis.

It should be appreciated however that embodiments of the present disclosure are not limited to any specific order of the scores or a corresponding magnitude of induration and/or erythema that the scores represent. Also, scores can be quantitative (e.g., without limitation, scores from 1 to 5), qualitative (e.g., without limitation, indications such that severe, moderate, absent, etc.) or they can be in the form of a combination of quantitative and/or qualitative indicators. In some embodiments, scores can be integers. In some embodiments, scores can be any types of numbers. In some embodiments, scores are two-, three-, or multi-dimensional scores representing more than one feature of the induration and/or erythema, and/or other properties of the subject's skin at the site of the injection. The number and types of scores can be selected based on purposes of the SARS-CoV-2 diagnosis and/or on the settings in which the diagnosis is performed. In some embodiments, a plurality of scores are generated, each representing or associated with a probability (or other measure) of exposure to SARS-CoV-2 (e.g. of a human subject having SARS-CoV-2 infection, having had exposure to SARS-CoV-2, or having been vaccinated against SARS-CoV-2).

In use, a device can be positioned such that a device's camera is facing a location of the injection and the camera is activated to acquire image information. The tool (e.g., a mobile app) executed on the device can generate notifications (e.g. audio, video, a combination thereof, etc.) informing the user when suitable number of images of appropriate quality are acquired for the testing. The app can generate various other notifications to a user, including without limitation notification(s) of when to take an image of the injection site, etc. In some implementations, the tool can provide instructions to the user regarding image acquisition. Once the image(s) are acquired, the tool can provide information generated based on the image processing, such as one or more scores (or other indicators) representative of the skin response. The scores, which can be generated by the device's processor (e.g., locally) or by a processor of a remote device, can display the score(s) on the device's user interface. In this way, the subject can view the score. Any other associated information can be displayed on conjunction with the score. In some implementations, however (e.g. when the tool is part of a telehealth system), the scores, and/or other information generated as a result of processing of the images of the skin injection site, are communicated by the device to a healthcare provider system rather than being displayed on the device such as a user device. In such embodiments, the system accessible by a healthcare provider can display the score(s) and related information (e.g. subject's characteristics, medical history, travel history, etc.) of a user interface of a suitable display device. In some implementations, a tool used in embodiments of the present disclosure can be part of a COVID tracking platform (e.g. contact tracing platform) or a platform for tracking various other infectious diseases.

In embodiments, images of the injection site on the subject's skin can be acquired prior to the injection of an immunogenic composition (to generate control image information) and after the injection. In this way, the processing of the image information can take into account the condition and characteristics of the subject's skin prior to the injection. In some embodiments, images are acquired 2, or 3, or 4 days after the injection. In some embodiments, images are acquired on days 2, 3, and 4 after the injection. Thus, the prediction regarding the subject's exposure to SARS-CoV-2 based on the subject's skin reaction can be determined using a progression of images, representing the development of the skin reaction from the day of the injection and onwards. Various other image information can be used for control, including information acquired from other subjects.

In some embodiments, a processor of the device used to acquire images of the subject's skin reaction at the injection site can be configured to process the acquired image information and to generate a prediction regarding whether or not the skin response is indicative of subject's prior exposure to SARS-CoV-2 or prior vaccination with the spike protein. Additionally or alternatively, the device, which is configured to communicate with other devices over a network, can transmit the acquired image information comprising at least one image of the subject's skin another computer device, e.g., a server. The server can analyze the received image information to generate a prediction. The server, which can be or can be part of a cloud storage platform, can have or can be associated with hardware memory storing digital images acquired from multiple subjects' device (which can be positioned at different, remote locations).

In embodiments, a machine learning classifier can be executed by a processor of a computing device used to acquire image information or of another suitable device or system. The machine learning classifier can be part of image analysis software executed by the processor. The classifier can employ features, generated based on a training set comprising a plurality of images of skin injection sites. Some or all of the images in the training set can be labeled with an indication of a corresponding severity of exposure to SARS-CoV-2 (or absence of the exposure), based on a visual inspection of the images and/or using diagnosis information confirmed using other techniques (e.g., a polymerase chain reaction (PCR) analysis). The training set of images can be updated as new skin images at an injection site are acquired. The training set of images can be stored on or in association with a server that can receive the images from various devices.

A suitable feature selection approach can be used to select features appropriate for analysis of images at the site of injection of an immunogenic composition. In some embodiments, the classifier can additionally employ features other than those extracted from the digital images of the injection site. The classifier can use supervised, unsupervised, and/or semi-supervised approach. A dimensionality reduction can be performed to reduce a number of features used in the analysis.

In embodiments, non-limiting examples of a classifier include a maximum likelihood classifier, an artificial neural network (e.g., a deep neural network or another type), a convolutional neural network (CNN), support vector machine (SVM), a Naive Bayes classifier, random forest, a decision tree, regression algorithm (e.g., linear, logistic, or multivariate regression), a K-means classifier, learning vector quantization (LVQ), self-organizing map (SOM), a deep learning classifier, and combinations thereof. In some embodiments, a classifier includes a combination of a plurality of classifiers. The classifier receives as an input at least one image of the subject's skin at the injection site, and generates an output comprising one or more scores indicating a probability of the subject having or having had a SARS-CoV-2 infection, or having been vaccinated.

An effective skin response is only obtained if a sufficient amount of the S protein remains at the site of injection, however, the size of some polypeptides may be so small that the polypeptide diffuses rapidly in the extracellular compartment at the site of injection resulting in a less effective skin response. Consequently, an aspect of the present invention is a method and kit wherein the immunogenic composition comprises either a homopolymer or a heteropolymer of the polypeptide, whereby the polypeptide does not diffuse freely in the extracellular compartment and is efficiently taken up by antigen-presenting cells at the site of injection.

A homopolymer of the polypeptide is to be understood in its usual meaning, i.e. a polymer formed by two or more identical polypeptides, whereas a heteropolymer may be formed by at least two different polypeptides, or formed by a polypeptide and a heterologous carrier molecule. The homopolymer may be formed by two or more copies of the polypeptide, such as 2 to 6 copies, 2 to 10 copies, or 2 to 20 copies.

An example of the synthesis of a homopolymer may be the introduction of one or more N-terminal cysteine residues in the polypeptide, thereby allowing the homopolymer to be formed as a result of intermolecular disulphide bridges. The synthesis of a heteropolymer may be carried out by coupling the polypeptide to another polypeptide, such as the Envelope (E) protein, Nucleocapsid (N) protein, or Membrane (M) protein or part thereof. Alternatively, the heteropolymer may be any of the E protein, N protein, or M protein. By the synthesis of polymers of the polypeptide the specific activity or potency will increase because the polypeptide will not diffuse freely in the extracellular compartment, whereby a smaller dose of the polypeptide is necessary to elicit an observable DTH reaction.

Other kinds of modifications of the polypeptide may be relevant in order to increase the activity of it. Such modifications may be post-translational modifications such as acylation, i.e. addition of a lipid moiety or glycosylation.

In the kit according to the invention the immunogenic composition comprises 0.5 to 75 μg of the polypeptide, such as 0.5 to 50 μg of the polypeptide, or 5 to 50 μg of the polypeptide. When the immunogenic composition comprises polymers of the polypeptide the same amounts are suitable.

In an embodiment of the invention, the amino acid sequence of the polypeptide comprises an amino acid sequence which has at least 95, 96, 97, 98 or 99% sequence identity to the amino acid sequence shown in SEQ ID NO: 2, which is the sequence of the S protein from SARS-CoV-2, or having 95, 96, 97, 98 or 99% sequence identity to the amino acid sequence of an immunologically equivalent variant of the polypeptide.

The polypeptide may also be a variant of the polypeptide with the amino acid sequence shown in SEQ ID NO: 2, in that the amino acid sequence of the variant is homologous to an analog or a subsequence of the amino acid sequence shown in SEQ ID NO: 2.

The term “sequence identity” is used here to illustrate the degree of identity between the amino acid sequence of a given polypeptide and the amino acid sequence shown in SEQ ID NO: 2. The amino acid sequence to be compared with the amino acid sequence shown in SEQ ID NO: 2 may be deduced from a DNA sequence, e.g. obtained by hybridization as defined above, or may be obtained by conventional amino acid sequencing methods. The degree of sequence identity is preferably determined on the amino acid sequence of a mature polypeptide, i.e. without taking any leader sequence into consideration. In an embodiment, the degree of sequence identity is at least 95%, at least 96%, at least 97%, at least 98% or even 99% with the amino acid sequence shown in SEQ ID NO: 2.

Each of the polypeptides may be characterized by specific amino acid and nucleic acid sequences. It is to be understood, however, that such sequences include analogs and variants produced by recombinant methods wherein such nucleic acid and polypeptide sequences have been modified by substitution, insertion, addition or deletion of one or more nucleotides in said nucleic acid sequences to cause the substitution, insertion, addition or deletion of one or more amino acid residues in the recombinant polypeptide. When the term DNA is used in the following, it should be understood that for the number of purposes where DNA can be substituted with RNA, the term DNA should be read to include RNA embodiments which will be apparent for the man skilled in the art.

In order to possess an ability of eliciting a DTH reaction, a polypeptide must be at least twelve amino acids long, at least fifteen amino acids, or at least 20 amino acids. The relevant functional parts of the polypeptide with respect to the ability of the polypeptide to elicit a DTH reaction are the lymphoid cell epitopes, i.e. the parts of the amino acid sequence that are recognized by lymphoid cells. These epitopes may either be linear or structural.

The injection of the polypeptide may lead to an undesired sensitization of the human subjects diagnosed for SARS-CoV-2 infection if the same human subject will be subjected to the skin test more than twice, or in extreme situations more than once. Consequently, an object of the present invention is a kit wherein the polypeptide has been modified in order to abolish or delete sensitizing epitopes, without abolishing the epitopes that are relevant with respect to the DTH reactions. This may be carried out by several methods well-known to the human subject skilled in the art. One method may be to modify the polypeptide by denaturing procedures, such as autoclaving or formaldehyde treatment. Another method may be to modify the nucleotide sequence encoding the polypeptide in such a way that the translated amino acid sequence lacks all or some of the sensitizing epitopes.

In the present context the wording “sensitizing epitopes” means epitopes that cause sensitization of a human subject when the skin test has been used for diagnostic purposes. These epitopes may be either B-cell epitopes or T-cell epitopes. Due to genetic variation, human subjects may be divided into responders and non-responders to a specific polypeptide based on their ability of raising a lymphoid cell immune response to the polypeptide. Thus, for some polypeptides, a skin test wherein only one polypeptide is present may give rise to false negative responses, i.e. negative responses even though the human subject is suffering from SARS-CoV-2 infection because the lymphoid cell immune system of the human subject has not been able to raise an immune response towards the polypeptide. Consequently, in an embodiment of the present invention, the immunogenic composition comprises at least two different polypeptides either separated, or as polymers as described above, all the polypeptides being as defined above.

An immunogenic composition according to the invention is a composition suitable for intradermal injection. Thus, an object of the invention is a method of vaccinating one or more selected human subjects of a population against SARS-CoV-2 and subsequently subjecting the population to diagnostic tests for an immune response, comprising vaccinating the human subjects with a vaccine, which comprises as its, effective component the above-discussed immunogenic agent, and subsequently subjecting the population to intradermal injection of an immunogenic composition containing a polypeptide with which lymphoid cells previously primed with S protein from SARS-CoV-2 are capable of reacting in vivo, whereby a positive skin response at the location of injection is indicative of the human subject having or had SARS-CoV-2 infection, or vaccination thereto, and a negative skin response at the location of injection is indicative of the human subject not having coronavirus.

### EXAMPLES

### Example 1: DTH Reactions in Human Subjects Vaccinated Against SARS-CoV-2

A total of 200 patients are treated with multiple intradermal injections of DNP-modified vaccine containing SARS-CoV-2 S protein mixed with BCG. Four vaccine dosage-schedules are tested sequentially. Patients are tested for DTH to S protein from SARS-CoV-2 (DNP-modified and unmodified). For each cellular skin test material, 0.15 ml was drawn into a 0.5 cc Lo-Dose® insulin syringe (Becton Dickinson) and injected intradermally into the ventral forearm, making sure that a wheal was raised by the injection. After 48 hours, each reaction is palpated to determine the boarders of the area of induration by running the index finger along the skin toward the skin test reaction until an edge was felt, and each edge is marked by a pen line. The distances between each of the opposite sets of two pen lines are measured. A positive response is defined as: maximum diameter of induration mm, which, as indicated above, is the standard criterion for positivity in assessing DTH to microbial antigens.

DTH data are analyzed by determining the maximum DTH response exhibited by each patient to each of the test reagents. For most patients DTH to S protein was tested at only one post-treatment time point. Certain patients will undergo a second DTH test after a median interval of 1.9-9.5 months. There should be a tight correlation between DTH responses elicited at the two time points; e.g. for DTH to S protein.

1. A method of eliciting an immune response in a human subject showing no signs or symptoms of an active SARS-CoV-2 infection comprising administering intra-dermally to the human subject an immunogenic composition comprising the Spike protein (S protein) of SARS-CoV-2 or a fragment thereof.

2. The method according to claim 1, further comprising measuring the magnitude of induration and erythema in the skin at the site of the injection.

3. The method according to claim 2, wherein the magnitude of induration is measured between twenty four to seventy-two hours after administration of the immunogenic composition.

4. The method according to claim 2, wherein the magnitude of the induration in the skin is measured by using a software tool executed by a hardware processor of a computer device, wherein the computer device is configured to acquire at least one image of the skin at the site of the injection.

5. The method according to claim 4, wherein the computer device is a mobile device.

6. The method according to claim 4, wherein the at least one image is analyzed at least in part by the hardware processor to automatically generate at least one indicator of a plurality of indicators each representing a respective probability of the human subject having SARS-CoV-2 infection, having had exposure to SARS-CoV-2, or having been vaccinated against SARS-CoV-2.

7. The method according to claim 6, wherein the plurality of indicators comprise a plurality of scores.

8. The method according to claim 1, wherein the fragment thereof is the S1 subunit of the S protein.

9. The method according to claim 1, wherein the fragment thereof is the S2 subunit of the S protein.

10. The method according to claim 1, wherein the fragment thereof is the receptor binding domain (RBD) of the S Protein.

11. The method according to claim 1, wherein the S protein comprises SEQ ID NO: 2.

12. The method according to claim 8 wherein the S1 subunit comprises amino acids 17-680 of SEQ ID NO: 2.

13. The method according to claim 9, wherein the S2 subunit comprises amino acids 727-1195 of SEQ ID NO: 2.

14. The method according to claim 10, wherein the receptor binding domain comprises amino acids 417-560 of SEQ ID NO:2.

15. The method according to claim 1, wherein the S protein or fragment thereof is administered as a fusion protein.

16. The method according to claim 1, wherein the S protein or fragment thereof is conjugated to a hapten.

17. The method according to claim 1, wherein the immunogenic composition comprises at least one excipient.

18. The method according to claim 17, wherein the at least one excipient comprises phosphate buffered saline (PBS) with 0.01% Polysorbate-20 and 0.5% phenol.

19. The method according to claim 1, wherein the S protein or fragment thereof is present in an amount of 5-50 μg/mL.

20. The method according to claim 1, wherein the immunogenic composition comprises one or more additional SARS-CoV-2 proteins selected from the group consisting of M protein, N protein and E protein.

21-25. (canceled)
